SEQUENCE LISTING (2 sequences)

SEQ ID NO: 1 - DNA, 2292 bp, Homo sapiens
  CDS (1)..(2289)
atgaggccggcggacttgctgcagctggtgctgctgctcgacctgcccagggacctgggcggaatggggtgttcgtctccaccctgcgagtgccatcaggaggaggacttcagagtcacctgcaaggatattcaacgcatccccagcttaccgcccagtacgcagactctgaagcttattgagactcacctgagaactattccaagtcatgcattttctaatctgcccaatatttccagaatctacgtatctatagatgtgactctgcagcagctggaatcacactccttctacaatttgagtaaagtgactcacatagaaattcggaataccaggaacttaacttacatagaccctgatgccctcaaagagctccccctcctaaagtccttggcattttcaaacactggacttaaaatgttccctgacctgaccaaagtttattccactgatatattctttatacttgaaattacagacaacccttacatgacgtcaatccctgtgaatgcttttcagggactatgcaatgaaaccttgacactgaagctgtacaacaatggctttacttcagtccaaggatatgatttctttgggacaaagctggatgctgtttacctaaacaagaataaatacctgacagttattgacaaagatgcatttggaggagtatacagtggaccaagcttgctggacgtgtctcaaaccagtgtcactgcccttccatccaaaggcctggagcacctgaaggaactgatagcaagaaacagctggactcttaagaaacttgcactttccttgagtttccttcacctcacacgggctgacctttcttacccaagccactgctgtgcttttaagaatcagaagaaaatcagaggaatccttgagtccttgatgtgtaatgagagcagtatcgagacgttgcgccagagaaaatctgtgaatgccttgaatagccccctccaccaggaatatgaagagaatctgggtgacagcattgttgggtacaaggaaaagtccaagttccaggatactcataacaacgctcattattacgtcttctttgaagaacaagaggatgagatcattggttttggccaggagctcaaaaacccccaggaagagactctacaagcttttgacagccattatgactacaccatatgtggggacagtgaagacatggtgtgtacccccaagtccgatgagttcaacccgtgtgaagacataatgggctacaagttcctgagaattgtggtgtggttcgttagtctgctggctctcctgggcaatgtctttgtcctgcttattctcctcaccagccactacaaactgaacgtcccccgctttctcatgtgcaacctggcctttgcggatttctgcatggggatgtacctgctcctcatcgcctctgtagacctctacactcactctgagtactacaaccatgccatcgactggcagacaggccctgggtgcaacacggctggtttcttcactgtctttgcaagcgagttatcggtgtatacgctgacggtcatcaccctggagcgctggtatgccatcaccttcgccatggccctggaccggaagatccgcctcaggcacgcatgtgccatcatggttgggggctgggtttgctgcttccttctcgccctgcttcctttggtgggaataagtagctatgccaaagtcagtatctgcctgcccatggacaccgagacccctcttgctctggcatatattgtttttgttctgacgctcaacatagttgccttcgtcatcgtctgctgctgttatgtgaagatctacatcacagtccgaaatccgcacaacccaggggacaaagataccaaaattgccaagaggatggctgtgttgatcttcaccgacttcacgtgcatggccccaatctcattctatgctgtgtcagcaattctgaacaagcctctcatcactgttagcaactccaaaatcttgctggtactcttctatccaattaactcctgtgccaatccattcctctatgctattttcaccaaggccttccagagggatgtgttcatcctactcagcaagtttggcatctgtaaacgccaggctcaggcataccgggggcagagggttcctccaaagaacagcactgatattcaggttcaaaaggttacccacgacatgaggcagggtctccacaacatggaagatgtctatgaactgattgaaaactcccatctaaccccaaagaagcaaggccaaatctcagaagagtatatgcaaacggttttgtaa

SEQ ID NO: 2 - protein, 763 aa, Homo sapiens [sequence omitted]

(19) United States
(12) Patent Application Publication
(10) Pub. No.: US 2006/0059588 A1
(43) Pub. Date: Mar. 16, 2006

(54) Production of transformed plants expressing thyroid stimulating hormone receptor

(76) Inventors: Min-Ho Shong, Daejon (KR); Sun Lee, Daejon (KR); Jae-Geun Jin, Seongnam-si (KR); Seok-Min Jin, Seongnam-si (KR)

Correspondence Address: JHK Law, P.O. Box 1078, La Canada, CA, 91012-1078, US

(21) Appl. No.: 10/520,258

(22) PCT Filed: Jul. 2, 2003
(86) PCT No.: PCT/KR03/01308

§ 371 Date: Jul. 21, 2005

(30) Foreign Application Priority Data
Jul. 2, 2002 (KR) 10-2002-0038064

(51) Int. Cl. A01H 1/00; C12N 15/82; C12N 15/87

(52) U.S. Cl. 800/288; 435/468

(57) ABSTRACT

The present invention relates to a method for preparing transformed plants expressing thyroid stimulating hormone receptor In particular, the present invention relates to a method for preparing transformed plants expressing (hTSHR) or (hTSHR-ECD) which comprises the steps of: (a) transforming plant cells with the following polynucleotide sequences: (i) a polynucleotide sequence encoding hTSHR or hTSHR-ECD; (ii) a promoter that functions in plant cells to cause the production of an RNA molecule operably linked to the polynucleotide sequence of (i); and (iii) a 3′-non-translated region that functions in plant cells to cause the polyadenylation of the 3′-end of said RNA molecule; (b) selecting transformed plant cells; and (c) obtaining transformed plant by regenerating said transformed plant cells, transformed plants and a method for preparing hTSHR or hTSHR-ECD.

### FIELD OF THE INVENTION

The present invention relates to a method for preparing transformed plants expressing thyroid stimulating hormone receptor. In particular, the present invention relates to a method for preparing transformed plants expressing thyroid stimulating hormone receptor (hTSHR) or thyroid stimulating hormone receptor-extracellular domain (hTSHR-ECD), transformed plants, and a method for preparing hTSHR or hTSHR-ECD.

### DESCRIPTION OF THE RELATED ART

Molecular farming is a technique producing recombinant proteins in plants. Recently, in the developed countries, the studies on the medical application of molecular farming have been performed. On account of the fact that recombinant proteins are extensively used as therapeutic agents of various diseases, it is expected that molecular farming may be an advantageous technique providing highly valuable and safe recombinant proteins.

Medical proteins prepared from transformed animals have a problem in long-term safety due to the risk of zoonosis such as Mad Cow Disease and are not much profitable considering the production cost. Furthermore, when prokaryotic expression system is used for producing medical proteins, proteins are not subject to a secondary modification and result in no therapeutic effect. However, recombinant proteins obtained from plants by using molecular farming technique have no risk of zoonosis, are able to be conveniently prepared in large amount, and are suitably secondary-modified to treat diseases promisingly. Such useful proteins can be provided economically through molecular farming.

Ten % of world population suffers from various autoimmune diseases such as rheumatoid arthritis, autoimmune thyroid disease, multiple sclerosis, insulin dependent diabetes mellitus, and autoimmune skin disease. As there are no known ways to treat autoimmune diseases radically, symptomatic treatment which cures diseases according to symptom and immune suppression therapy over long term are generally employed.

It is expected that oral tolerance suppressing autoimmune reaction by induction of immunological tolerance through nasal oral or cavity administration of autoantigen may be applicable to treatment of human autoimmune disease after animal test in several years for treating and preventing such autoimmune disease (U.S. Pat. No. 5,733,547).

A technical problem to be solved for clinical application of oral tolerance is how to obtain efficiently a large amount of autoantigen to be used in treatment. That is because the essential feature of oral tolerance is to administer high-dose of autoantigen through oral or nasal cavity, which is inevitably required.

### DETAILED DESCRIPTION OF THIS INVENTION

The present inventors have made attempts to develop a novel system which provides a large amount of thyroid stimulating hormone receptor (hTSHR) elucidated as a human autoantigen. As a result, the present inventors have prepared plants transformed with hTSHR or hTSHR-ECD gene successfully and found that hTSHR or hTSHR-ECD produced from transformants has high antigenicity.

Accordingly, it is an object of this invention to provide a method for preparing transformed plants expressing hTSHR or hTSHR-ECD.

It is another object of this invention to provide transformed plants.

It is still another object of this invention to provide a method for preparing hTSHR or hTSHR-ECD.

In one aspect of this invention, there is provided a method for preparing transformed plants expressing thyroid stimulating hormone receptor (hTSHR) or thyroid stimulating hormone receptor-extracellular domain (hTSHR-ECD), which comprises the steps of:
(a) transforming plant cells with the following polynucleotide sequences: (i) a polynucleotide sequence encoding hTSHR or hTSHR-ECD; (ii) a promoter that functions in plant cells to cause the production of an RNA molecule operably linked to the polynucleotide sequence of (i); and (iii) a 3′-non-translated region that functions in plant cells to cause the polyadenylation of the 3′-end of said RNA molecule;
(b) selecting transformed plant cells; and
(c) obtaining transformed plant by regenerating said transformed plant cells.

In another aspect of this invention, there is provided a transformed plant which is prepared by the above method and expresses hTSHR or hTHSR-ECD.

According to this invention, plants transformed with a full length gene of autoantigen or its portion which involves in autoimmune thyroid disease are prepared and from them, a large amount of human thyroid stimulating hormone receptor (hTSHR), a representative human autoantigen, are provided for diagnosis and treatment.

A human autoantigen, hTSHR, has been proved to be an autoantigen causing hyperthyroidism. The antibody against hTHSR stimulates thyroid stimulating hormone receptor expressed on cell membrane of thyroid, which in turn increases the production of thyroid hormone and hyperthyroidism is finally developed, like the case of thyroid stimulating hormone. Therefore, the detection of IgG antibody against hTSHR could be applied to diagnosis of hyperthyroidism, and the mass production of hTSHR would enable hTSHR to be used in oral tolerance therapy.

hTSHR antibody found in patients is known not to bind to recombinant protein expressed in prokaryotic expression system, so that hTSHR prepared from E. coli is useless for detecting IgG of patients. This suggests that hTSHR expressed in prokaryote requires secondary modification such as glycosylation to function as an immunogen. Consequently, a eucaryotic expression system should be employed for the application of hTSHR as diagnostic antigen or to oral tolerance therapy. In addition, it is found that hTSHR expressed in an expression system derived from vaccinia virus is not valuable because of low binding affinity to antibody.

The present method for preparing human autoantigen from transformed plants is directed to the mass production of human proteins in plant and could be a pivotal technology for the next generation-drug development (particularly, for producing human autoantigen used in the treatment of autoimmune disease).

In the present invention, hTSHR or hTSHR-ECD corresponding to the extracellular domain of intact hTSHR is used. hTSHR-ECD is a significant region for hTSHR to function as antigen (GS Seetharamaiah, et al., Requirement of glycosylation of the human thyrotropin receptor ectodomain for its reactivity with autoantibodies in patients' sera, J. Immunol., 158:2798-2804(1997)).

The nucleotide sequence of hTSHR or hTSHR-ECD used herein includes the sequences known in the art as well as modified suitably for expression in plant cell. The exemplified hTSHR nucleotide sequence used in the present invention is set forth in SEQ ID NO:1 and the exemplified hTSHR-ECD nucleotide sequence corresponds to the polynucleotide spanning nucleotide 1-1254 of SEQ ID NO:1 which encodes the amino acid sequence 1-418 of SEQ ID NO:2. The modification of the nucleotide sequence for efficient expression in plants can be achieved by various manipulations such as adjusting GC content, introducing proper codon usage preferred in plant and removing intron-like sequence (Kusnadi et al., Biotechnol. Bioeng. 56:473-484(1997); and WO 9116432).

According to a preferred embodiment, promoters used conventionally for transformation of plant in the art may be used, including the cauliflower mosaic virus (CaMV) 35S promoter, nopaline synthase (nos) promoter, the Figwort mosaic virus 35S promoter, the sugarcane bacilliform virus promoter, the commelina yellow mottle virus promoter, the light-inducible promoter from the small subunit of the ribulose-1,5-bis-phosphate carboxylase (ssRUBISCO), the rice cytosolic triosephosphate isomerase (TPI) promoter, the adenine phosphoribosyltransferase (APRT) promoter of Arabidopsis, the rice actin 1 gene promoter, and the mannopine synthase and octopine synthase promoters.

The 3′-non-translated region suitable in this invention may include that from the nopaline synthase gene of Agrobacterium tumefaciens (nos 3′ end) (Bevan et al., Nucleic Acids Research, 11(2):369-385(1983)), that from the octopine synthase gene of Agrobacterium tumefaciens, the 3′-end of the protease inhibitor I or II genes from potato or tomato, the CaMV 35S terminator.

The transformation of plant cells may be carried out according to the conventional methods known one of skill in the art, including electroporation (Neumann, E. et al., EMBO J., 1:841(1982)), particle bombardment (Yang et al., Proc. Natl. Acad. Sci., 87:9568-9572(1990)) and Agrobacterium-mediated transformation (U.S. Pat. Nos. 5,004,863, 5,349,124 and 5,416,011). Among them, Agrobacterium-mediated transformation is the most preferable. Agrobacterium-mediated transformation is generally performed with leaf disks and other tissues such as cotyledons and hypocotyls. This method is the most efficient in dicotyledonous plants.

The selection of transformed cells may be carried out with exposing the transformed cultures to a selective agent such as a metabolic inhibitor, an antibiotic and herbicide. Cells which have been transformed and have stably integrated a marker gene conferring resistance to the selective agent will grow and divide in culture. The exemplary marker includes, but not limited to, a glyphosphate resistance gene and a neomycin phosphotransferase (nptII) system.

The development or regeneration of plants from either plant protoplasts or various explants is well known in the art. The resulting transgenic rooted shoots are planted in an appropriate plant growth medium. The development or regeneration of plants containing the foreign gene of interest introduced by Agrobacterium may be achieved by methods well known in the art (U.S. Pat. Nos. 5,004,863, 5,349,124 and 5,416,011).

Meanwhile, the present inventors have made attempts to develop novel transformed plants such as Nicotiana tabacum, Cucumis melo, Curcumis sativa, Citrullus vulgaris and Brassica campestris and as a result, have established the most efficient methods for the transformation of certain plant. Such methods have been filed for patent application (PCT/KR02/01461, PCT/KR02/01462 and PCT/KR02/01463) which is incorporated herein by reference.

The method of the present invention is applicable to various plants, preferably, Nicotiana tabacum, Cucumis melo, Curcumis sativa, Citrullus vulgaris and Brassica campestris.

In the present invention, it is preferred to use Agrobacterium system for transformation, more preferably, Agrobacterium tumefaciens-binary vector system.

The example of this invention employing Agrobacterium transformation system comprises the steps of: (a′) inoculating an explant material from the plant with Agrobacterium tumefaciens harboring a vector, which is capable of inserting into a genome of a cell from the plant and contains the following nucleotide sequences: (i) a polynucleotide sequence encoding hTSHR or hTSHR-ECD; (ii) a promoter that functions in plant cells to cause the production of an RNA molecule operably linked to the polynucleotide sequence of (i); and (iii) a 3′-non-translated region that functions in plant cells to cause the polyadenylation of the 3′-end of said RNA molecule; (b′) regenerating the inoculated explant material on a regeneration medium to obtain regenerated shoots; (c′) culturing the regenerated shoots on a rooting medium to obtain a transformed plant.

In the example, the explant for transformation includes any tissue derived from seed germinated. It is preferred to use cotyledon and hypocotyl and the most preferred is cotyledon. Seed germination may be performed under suitable dark/light conditions using an appropriate medium. Transformation of plant cells derived is carried out with Agrobacterium tumefaciens harboring Ti plasmid (Depicker, A. et al., Plant cell transformation by Agrobacterium plasmids. In Genetic Engineering of Plants, Plenum Press, New York (1983)).

More preferably, binary vector system such as pBin19, pRD400 and pRD320 is used for transformation (An, G. et al., Binary vectors” In Plant Gene Res. Manual, Martinus Nijhoff Publisher, New York (1986)). The binary vector useful in this invention carries: (i) a promoter capable of operating in plant cell; (ii) a structural gene operably linked to the promoter; and (iii) a polyadenylation signal sequence. The vector may alternatively further carry a gene coding for reporter molecule (for example, luciferase and β-glucuronidase). Examples of the promoter used in the binary vector include but not limited to cauliflower mosaic Virus 35S promoter, 1′ promoter, 2′ promoter and promoter nopaline synthetase (nos) promoter.

Inoculation of the explant with Agrobacterium tumefaciens involves procedures known in the art. Most preferably, the inoculation involves immersing the cotyledon in the culture of Agrobacterium tumefaciens to coculture. Agrobacterium tumefaciens is infected into plant cells.

The explant transformed with Agrobacterium tumefaciens is regenerated in a regeneration medium, which allows successfully the regeneration of shoots. The transformed plant is finally produced on a rooting medium by rooting of regenerated shoots.

The transformed plant produced according to the present invention may be confirmed using procedures known in the art. For example, using DNA sample from tissues of the transformed plant, PCR is carried out to elucidate exogenous gene incorporated into a genome of the transformed plant. Alternatively, Northern or Southern Blotting may be performed for confirming the transformation (Maniatis et al., Molecular Cloning, A Laboratory Manual, Cold Spring Harbor Laboratory, Cold Spring Harbor, N.Y. (1989)).

According to a preferred embodiment, the step of recovering hTSHR or hTHSR-ECD from transformed plants is further included. hTSHR or hTHSR-ECD may be provided from the tissues derived from various transformed organs (e.g., stem, leave, root, fruit and seed, etc) and be obtained by purifying the extracts of the tissues.

In this invention, purification methods conventionally used in the art may be employed. For example, various methods including solubility fractionation by use of ammonium sulfate or PEG, size differential filtration and column chromatography (based on size, net surface charge, hydrophobicity or affinity) are available and usually the combination of the methods is used for purification.

Therefore, according to another embodiment, this invention provides a method for preparing thyroid stimulating hormone receptor (hTSHR) or thyroid stimulating hormone receptor-extracellular domain (hTSHR-ECD), which comprises the steps of: (a) transforming plant cells with the following polynucleotide sequences: (i) a polynucleotide sequence encoding hTSHR or hTSHR-ECD; (ii) a promoter that functions in plant cells to cause the production of an RNA molecule operably linked to the polynucleotide sequence of (i); and (iii) a 3′-non-translated region that functions in plant cells to cause the polyadenylation of the 3′-end of said RNA molecule; (b) selecting transformed plant cells; (c) obtaining transformed plant by regenerating said transformed plant cells; and (d) recovering hTSHR or hTHSR-ECD from said transformed plant.

At present, the molecular farming with transformed plants and edible vaccines is considered plausible as technologies to provide novel therapeutic agents.

As this invention employs a eucaryote-expression system, the final products, hTHSR or hTHSR-ECD, are very likely to be secondarily modified (e.g., high antigenicity) and the convenient cultivation of transformed plants will allow to provide the mass production of human autoantigen, hTHSR or hTHSR-ECD. It could be appreciated that hTHSR or hTHSR-ECD prepared by this invention is applicable to oral tolerance therapy.

### BRIEF DESCRIPTION OF THE DRAWINGS

FIG. 1 shows a plant-expressing cassette of human thyroid stimulating hormone receptor gene (tshr);

FIG. 2 shows a plant-expressing cassette of thyroid stimulating hormone receptor-extracellular domain gene (tshr-ecd);

FIG. 3 shows the tshr or tshr-ecd gene cloned in plant-transformation vector pRD400;

FIG. 4 is a photograph showing the PCR result of the transformed tshr gene in plants transformed with tshr;

FIG. 5 is a photograph showing the PCR result of the transformed tshr-ecd gene in plants transformed with tshr-ecd;

FIG. 6 is a photograph showing the result of Western Blotting demonstrating the expression of TSHR in transformed plants; and

FIG. 7 is a photograph showing the result of ELISA analysis of TSHR produced in transformed plants using IgGs obtained from Grave's disease patient.

The following specific examples are intended to be illustrative of the invention and should not be construed as limiting the scope of the invention as defined by appended claims.

### EXAMPLES

### Example I

### Cloning of tshr Gene

From the known human tshr cDNA information registered in GenBank (http://www.ncbi.nlm.nih.gov/) (XM-056624, XM-041159, XM-041157, M73747 and BC009237), cDNA nucleotide sequence was obtained. The full length of human tshr gene was amplified by RT-PCR and cloned into TA vector. The insertion of the human tshr gene was then confirmed by sequencing.

i) Cloning of Full Length of tshr Gene

Firstly, a pair of primers designed on the basis of the nucleotide sequence of tshr gene searched in GenBank database was synthesized for PCR in order to subclone the full length of tshr gene (about 2.3 kb) into the plant-expression cassette of a vector. The primer for 5′-flanking region was designed to have a start codon of tshr gene and BamHI recognition site for cloning into cassette (5′-AAGGATCCC ATG AGG CCG GCG GAC-3′), and the primer for 3′-flanking region was designed to include a stop codon and BamHI recognition site for cloning into cassette (5′-ATGGATCC TTA CAA AAC CGT TTG CAT-3′).

25 μl of PCR mixture was prepared containing 1.25 unit Taq DNA polymerase (Boehringer Mannheim), 2.5 μl of 10× buffer (Boehringer Mannheim), 2 μl of 2.5 mM DNTP, 0.25 μl of 100 pM primers and 50 ng of DNA including tshr gene. The PCR was conducted under the following conditions: pre-denaturation at 95° C. for 2 min followed by 30 cycles of annealing at 55° C. for 1 min, extension at 72° C. for 1 min and denaturation at 92° C. for 1 min; followed by final extension at 72° C. for 10 min. The amplified products were kept at 4° C. for analysis and electrophoresed on 0.8% TAE agarose gel.

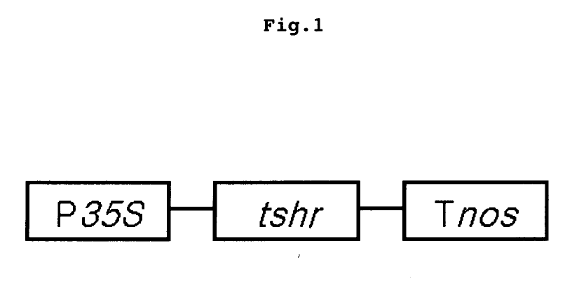

The tshr gene was eluted and obtained from the corresponding band. In amplified DNA, tshr gene has the nucleotide sequence as set forth in SEQ ID No:1. The tshr gene fragment purified with glass milk was digested with suitable restriction enzymes and inserted into the binary vector pRD400 for plant transformation containing plant-expression cassette (Raju et al., Gene 211:383-384(1992)) digested with the same restriction enzymes. A cassette for plant expression of tshr gene in FIG. 1 was constructed.

ii) Cloning of Portion of tshr Gene

Firstly, a pair of primers designed on the basis of the nucleotide sequence of tshr gene searched in GenBank database was synthesized for PCR in order to subclone into the cassette of the plant-expression vector the gene (tshr-ecd, about 1.24 kb) that encodes an extracellular domain of TSHR displayed on the outside of human cells.

The primer for 5′-flanking region was designed to include a start codon of tshr gene and BamHI recognition site for cloning into cassette (5′-AAGGATCCC ATG AGG CCG GCG GAC-3′), and the primer for 3′-flanking region was designed to amplify a nucleotide sequence from start point of tshr gene to around nucleotide 1239 wherein the extracelluar domain is encoded, and to have additional stop codon and BamHI recognition site for cloning into cassette (5′-ATGGATCC TTA GCC CAT TAT GTC TTC-3′).

25 μl of PCR mixture was prepared containing 1.25 unit Taq DNA polymerase (Boehringer Mannheim), 2.5 μl of 10× buffer (Boehringer Mannheim), 2 μl of 2.5 mM dNTP, 0.25 μl of 100 pM primers and 50 ng of DNA including tshr-ecd. The PCR was conducted under the following conditions: pre-denaturation at 95° C. for 2 min followed by 30 cycles of annealing at 55° C. for 1 min, extension at 72° C. for 1 min and denaturation at 92° C. for 1 min; followed by final extension at 72° C. for 10 min. Amplified products were analyzed by electrophoresis on 0.8% TAE agarose gel at the constant temperature of 4° C. The desired tshr DNA fragment eluted and obtained from the corresponding band.

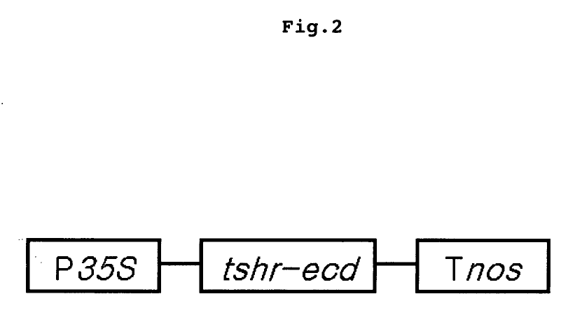

The tshr-ecd fragment purified with glass milk was digested with suitable restriction enzymes and inserted into binary vector pRD400 for plant transformation (Raju et al., Gene 211:383-384(1992) containing plant-expression cassette digested with the same restriction enzymes. A cassette for plant-expression of tshr-ecd in FIG. 2 was constructed.

### Example II

### Transformation of Plant

i) Infection of Agrobacterium tumefaciens GV3101

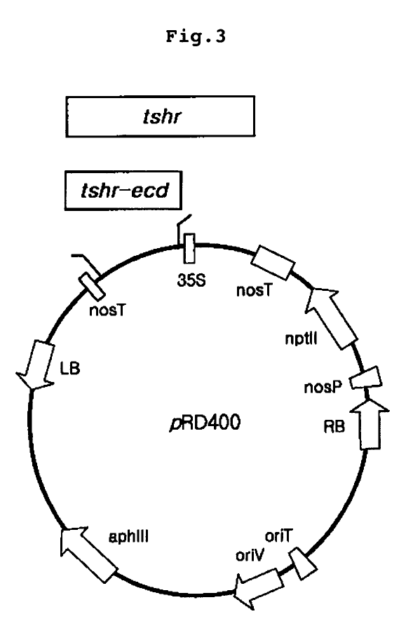

pRD400-tshr and pRD400-tshr-ecd (FIG. 3) of Example I obtained by cloning into the binary vector for plant transformation, pRD400, was introduced respectively into Agrobacterium tumefaciens (Agrobacterium tumefaciens GV3101(mp90); Plant-cell-rep., 15(11)799-803(1996)) by means of conjugation. To select Agrobacterium tumefaciens harboring the vector, the incubated mixture for conjugation was spread on LB solid medium containing 50 mg/L of kanamycin and 30 mg/L of gentamicin and incubated for 2 days at 28° C. The selected Agrobacterium tumefaciens containing desired gene was inoculated into super broth (BHI medium, pH 5.6), incubated for 2 days at 28° C. and used for infection of plant.

ii) Transformation of Cucumis melo

The seeds of Cucumis melo sterilized were seeded for obtaining cotyledons. The cotyledons were collected in a manner that their growth points were completely removed. Agrobacterium tumefaciens transformed with pRD400-tshr or pRD400-tshr-ecd was incubated for 18 hr at 28° C. in super broth containing 100 μM acetosyringone (37 g/l brain heart infusion broth(Difco) and 0.2% sucrose, pH 5.6), and then the resulting medium was diluted 20-fold with inoculation medium. The above inoculation medium (pH 5.6) contains MSB5 (Murashige & Skoog medium including Gamborg B5 vitamins), 3.0% sucrose, 0.5 g/L of MES [2-(N-Morpholino)ethanesulfonic acid Monohydrate], 6.0 mg/L of kinetin, 1.5 mg/L of IAA (indole-3-acetic acid), 1.0 mg/L of CuSO₄.5H₂O, 100 μM acetosyringone and 5% DMSO.

Thereafter, the cotyledon was immersed in 40 ml of the inoculation medium and incubated for 20 min. Then, the cotyledon was transferred to a coculturing medium (MSBS, 3.0% sucrose, 0.5 g/L of MES, 6.0 mg/L of kinetin, 1.5 mg/L of IAA, 1.0 mg/L of CuSO₄.5H₂O, 0.6% agar, 100 μM acetosyringone and 5% DMSO). The cotyledon was then cocultured under dark culture condition (26±1° C., 24 hrs night) for 3 days. After coculturing, the cotyledon was placed on a selection medium (MSB5, 3.0% sucrose, 0.5 g/L of MES, 6.0 mg/L of kinetin, 1.5 mg/L of IAA, 1.0 mg/L of CuSO₄.5H₂O, 0.6% agar, 100 mg/L of kanamycin and 500 mg/L of carbenicillin, pH 5.6) and light-cultured at 26±1° C. and 4,000 lux under 16 hr light condition for 3 days to induce generation of shoots.

The elongated shoots were transferred to a rooting medium (MSB5, 3.0% sucrose, 0.5 g/L of MES, 0.1 mg/L of NAA (α-naphtalene acetic acid), 1.0 mg/L of CuSO₄.5H₂O, 0.6% agar, 100 mg/L of kanamycin and 500 mg/L of carbenicillin, pH 5.6) and cultured for 2 weeks. The shoots with roots, which were considered to be transformed, were selected.

iii) Transformation of Curcumis sativa

The seeds of Curcumis sativa sterilized were seeded for obtaining cotyledons. The cotyledons were collected in a manner that their growth points were completely removed. Agrobacterium tumefaciens transformed with pRD400-tshr or pRD400-tshr-ecd was incubated in the same manner as described in i). The sections of the cotyledon were immersed for 10 min in the inoculation medium containing Agrobacterium in the same manner as described in ii).

Thereafter, the cotyledon was cultured in a coculturing medium (MSB5 containing 2 mg/L of BAP and 0.01 mg/L of NAA) under light culture condition at 26° C. for 2 days and then was cocultured with Agrobacterium tumefaciens at 4° C. for 4 days. After coculturing, the cotyledon was placed on a selection medium containing MSB5, 3.0% sucrose, 0.5 g/L of MES, 0.4% phytagel, 2 mg/L of BAP, 0.01 mg/L of NAA, 500 mg/L of carbenicillin and 100 mg/L of kanamycin and cultured at 26±1° C. and 8,000 lux under 16 hr light/B hr dark condition.

Then, the regenerated shoots were transferred to a rooting medium (containing 0.01 mg/L of NAA, 100 mg/L of kanamycin and 0.4% agar) and cultured at 26±1° C. and 8,000 lux under 16 hr light/8 hr dark condition. The shoots with roots considered to be transformed were analyzed by the method described in Example below.

iv) Transformation of Citrullus vulgaris

The seeds of Citrullus vulgaris sterilized were seeded for obtaining cotyledons. The cotyledons were collected in a manner that their growth points were completely removed. Agrobacterium tumefaciens transformed with pRD400-tshr or pRD400-tshr-ecd was incubated in the same manner as described in i). The cotyledon was immersed for 10 min in the inoculation medium containing Agrobacterium in the same manner as described in ii). Thereafter, the cotyledon was placed on a coculturing medium (4.04 g/L of MSB5, 3.0% sucrose, 0.5 g/L of MES and 0.6% agar, pH 5.6) and cultured under 16-hour light culture condition at 25±1° C. and 4,000 lux for 2 days. Cultured cotyledon was placed on the medium (MSB5, 2 mg/L of BAP, 3.0% sucrose, 0.5 g/L of MES, 0.4% phytagel, 500 mg/L of carbenicillin and 200 mg/L of kanamycin, pH 5.6) and cultured at 25° C.±1° C. for 7 days. Following the incubation for 4 weeks, the shoots were selected.

v) Transformation of Brassica campestris

The seeds of Brassica campestris sterilized were seeded for obtaining petiole. The petioles were collected in a manner that their growth points were completely removed. Agrobacterium tumefaciens transformed with pRD400-tshr or pRD400-tshr-ecd was incubated in the same manner as described in i). The petiole was immersed for 10 min in the inoculation solution containing Agrobacterium in the same manner as described in ii). Thereafter, the petiole was cultured in a coculturing medium (MSB5, 3% sucrose, 1 mg/L of 2,4-D and 6.5 g/L of agar power, pH 5.8) at 25° C. for 2 days and subsequently at 4° C. for 4 days.

To select the transformed Brassica campestris, the petiole was transferred to a selection medium (MSB5, 3% sucrose, 5 g/L of MES, 2 mg/L of BAP, 0.01 mg/L of NAA, 20 mg/L of kanamycin, 500 mg/L of Psedopen and 6.5 g/L of agar power, pH 5.8) and cultured at 25° C. for 2 weeks under 16-hr light/8-hr dark condition. Two weeks after, The root for shoot was induced in a rooting meduium (pH 5.8) containing MSB5, 3.0% sucrose, S g/L of MES, 0.1 mg/L of NAA, 20 mg/L of kanamycin 500 mg/L of Pseudopen and 6.5 g/L of agar.

vi) Transformation of Nicotiana tabacum

The sterilized seeds of Nicotiana tabacum were seeded and cultivated in sterilized condition over 2 weeks for obtaining young leaves. Agrobacterium tumefaciens transformed with pRD400-tshr or pRD400-tshr-ecd was incubated fin the same manner as described in i of Example 2 and then mixed with the inoculation medium as in ii of Example 2. The fragments of young leaf with a size of 0.5-1 cm² were immersed for 10-15 min in the inoculation medium and then transferred to a coculturing medium (MSB5, 3.0% sucrose, 0.5 g/L of MES, 1.0 mg/L of BAP, 0.1 mg/L of NAA and 0.6% agar, pH 5.8).

The fragment was cocultured under dark culture condition (26±1° C., 24 hrs night) for 2 days. After coculturing, in order to form shoots by regeneration, the fragment was placed on a selection medium (MSB5, 3.0% sucrose, 0.5 g/L of MES, 1.0 mg/L of BAP, 0.1 mg/L of NAA, 0.6% agar, 100 mg/L of kanamycin and 500 mg/L of carbenicillin, pH 5.6) and cultured at 26±1° C. and 4,000 lux for 2 weeks under 16-hr light condition. The elongated shoots were transferred to a rooting medium (MSBS, 3.0% sucrose, 0.5 g/L of MES, 0.01 mg/L of NAA, 0.6% agar, 100 mg/L of kanamycin and 500 mg/L of carbenicillin, pH 5.6) and cultured for 2 weeks. The shoots with roots, which were considered to be transformed, were selected.

### Example III

### Verification on Transformation of Plant by PCR

The transformants in Example II were verified as described below:

Using 10 mg of the shoots that were selected to be transformed, a genomic DNA for PCR analysis was obtained according to the method described by Edwards K., et al. (Nucleic Acids Research, 19: 1349(1991)) and then PCR analysis was performed.

The primer set for PCR analysis of plant transformed with pRD400-tshr is corresponding to nucleotide sequence of tshr gene: forward primer, 5′-AAGGATCCC ATG AGG CCG GCG GAC-3′; and reverse primer, 5′-ATGGATCC TTA CAA AAC CGT TTG CAT-3′.

The primer set for PCR analysis of plant transformed with pRD400-tshr-ecd is corresponding to nucleotide sequence of tshr-ecd gene: forward primer, 5′-AAGGATCCC ATG AGG CCG GCG GAC-3′; and reverse primer, 5′-ATGGATCC TTA GCC CAT TAT GTC TTC-3′.

The PCR amplification was conducted using Taq polymerase according to the following thermal conditions: pre-denaturation at 96° C. for 2 min followed by 35 cycles of annealing at 55° C. for 1 min, extension at 72° C. for 2 min and denaturation at 94° C. for 1 min; followed by final extension at 72° C. for 10 min. Amplified products were analyzed by electrophoresis on 1.0% agarose gel.

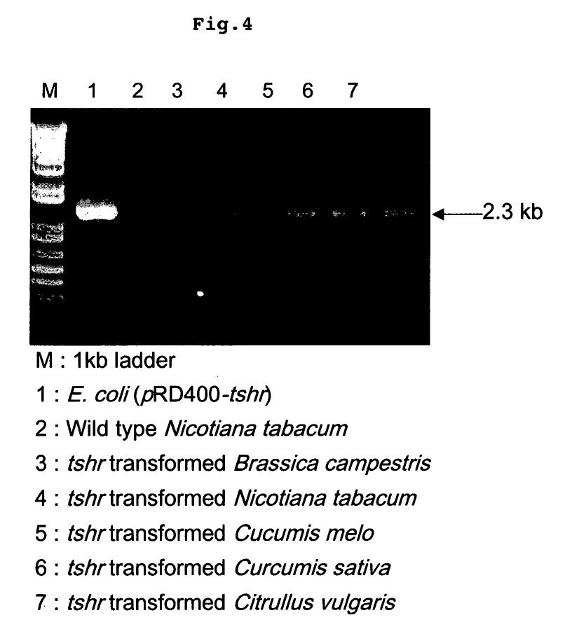

In FIG. 4, lane M shows 1 kb ladder, lane 1 represents PCR product of positive standard plasmid containing gene, pRD400-tshr, lane 2 represents PCR product of wild-type Nicotiana tabacum and lanes 3,4,5,6 and 7 represent PCR products of selected Brassica campestris, Nicotiana tabacum, Cucumis melo, Citrullus vulgaris and Curcumis sativa, respectively. As shown in FIG. 4, the band corresponding to tshr gene (2.3 kb) is observed in each lane, which indicated the successful transformation of plant in Example described above.

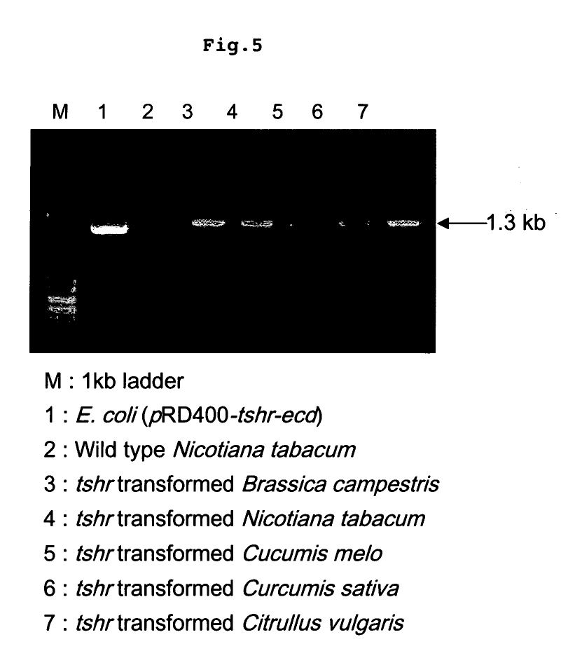

In FIG. 5, lane M shows 1 kb ladder, lane 1 represents PCR products of positive standard plasmid containing gene, pRD400-tshr-ecd, lane 2 represents PCR product of wild-type Nicotiana tabacum and lanes 3,4,5,6 and 7 represent PCR products of selected Brassica campestris, Nicotiana tabacum, Cucumis melo, Citrullus vulgaris and Curcumis sativa, respectively. As shown in FIG. 5, the band corresponding to tshr-ecd gene (1.3 kb) is observed in each lane, which indicated the successful transformation of plant in above Example.

### Example IV

### Verification of TSHR or TSHR-ECD in Plant Transformants by Western Blotting

2.5 ml of extraction buffer pre-prepared (5 ml of 100 mM Tris-Cl, pH 7.5, 40 μl of 500 mM EDTA, pH 8.0, 1.5 ml of 1 mg/ml leupeptin, 600 μl of 5 mg/ml BSA, 3 ml of 1 mg/ml DTT and 50 μl of 30 mg/ml PMSF (stock solution; 0.003 g PMSF in 10 μl IPA) added just before use) were added to 1 g of the chopped leaves of transformants and then the leaves were ground finely in a mortar. The extract was centrifuged at 12,000 rpm and 4° C. for 30 min, the supernatant was transferred to a new tube and stored on ice. The quantification of proteins in plant transformants was performed in accordance with Bradford method in a manner that dye (protein assay kit, Bio-Rad) was added to the extract and the absorbance at 595 nm was measured with UV-spectrophotometer, followed by determining the protein amount with reference to the standard curve of bovine serum albumin. Then, the supernatant samples with the same amount were electrophoresed on 8% polyacrylamide gel

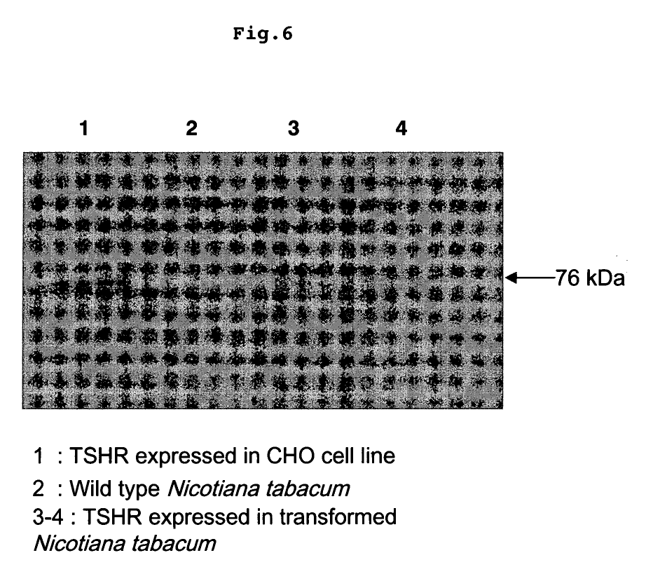

The protein band appearing by polyacrylamide gel electrophoresis was transferred to PVDF membrane and then the primary antibody (anti TSHR-rabbit, 1:1000 dilution, Santa Cruz Biotechnology, INC) was added to PVDF membrane and incubated for 1 hr. After incubation, the membrane was washed and incubated with the secondary antibody (rabbit-goat HRP, 1:1000 dilution, Santa Cruz Biotechnology, INC) for 1 hr and washed. Then, the color development was allowed with 4-chloro-1-naphtol (4-CN). The bands showing the expected size of TSHR, i.e., about 76 kDa were observed, so that the existence of TSHR in transformants was verified (see FIG. 6).

### Example V

### Analysis of TSHR and TSHR-ECD Expressed in Plant Transformants by ELISA

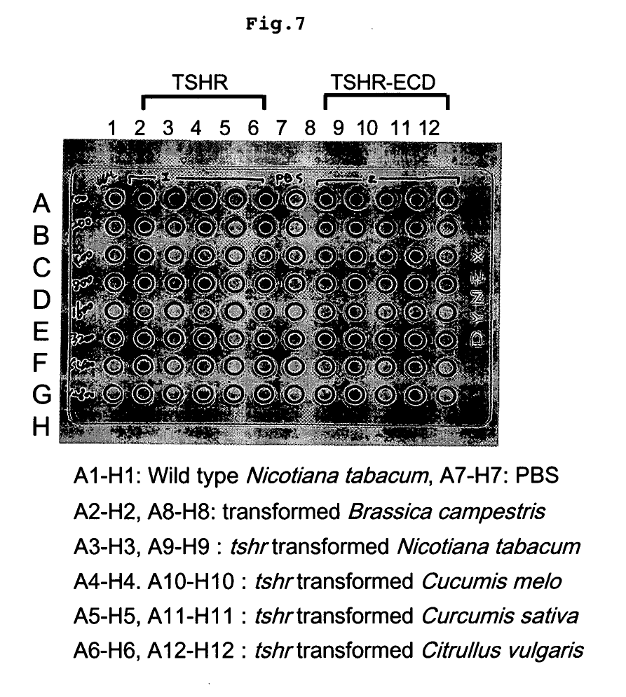

One ml of pre-prepared PBS (20 mM potassium phosphate, 150 mM NaCl pH 7.4) was added to 1 g of the chopped leaves of transformants and the leaves were ground finely in a mortar. The extract was centrifuged at 10,000 rpm and 4° C. over 5 min, the supernatant was transferred to a new tube and stored on ice. 50 μl of PBS was added to each of 96 wells in a ELISA plate. As shown in FIG. 7, the extract of wild-type Nicotiana tabacum as a negative control and the extracts of Brassica campestris, Nicotiana tabacum, Cucumis melo, Citrullus vulgaris and Curcumis sativa which were transformed with pRD400-tshr or pRD400-tshr-ecd were added to the corresponding wells in the ELISA plate and allowed to be coated on well in moisture chamber at 4° C.

Each of coated wells in plate was washed 4-5 times with 200 μl of washing buffer (PBS+0.2% Tween20) and then 50 μl of dilution buffer (washing buffer+5% skim milk) at 4° C. was added, followed by incubation in moisture chamber for blocking at 37° C. for 1 hr. After blocking, each well was washed with 200 μl of washing buffer 4-5 times. Serum of Grave's disease patient was serially diluted to 1/100, 1/200, 1/400, 1/800, 1/1600, 1/3200, 1/6400 and 1/12800 and 50 μl of the dilute of each concentration was added to from lanes A to H as in FIG. 7 and allowed to stand at 4° C. for 2 hrs. As above, each of coated wells in plate was washed with 200 μl of washing buffer and 300 μl of blocking buffer (PBS buffer+1% BBA, 5% sucrose and 0.05% NaN₃) at 4° C. was added, followed by incubated in moisture chamber for blocking at 4° C. for 1 hr. After blocking, each well was washed with 200 μl of washing buffer three times. Ten μl of diluted IgG conjugate (peroxidase labeling, 1/1000 dilution) were added and allowed to stand at 4° C. for 1 hr.

Thereafter, the wells were washed with washing buffer and ABST peroxidase substrate (KPL corp. U.S.A.) was added, followed by incubation for 30 min. The reaction was stopped with 50 μl of the stop buffer. The absorbance at 405 nm was measured with ELISA reader (TECAN sunrise). The result of analysis of recombinant proteins expressed in plants transformed with tshr gene, as shown in FIG. 7, shows clear reaction even in about 1/5000-diluted samples, demonstrating that the functional TSHRs were expressed in plant transformants.

As described above, the present invention provides a method for preparing transformed plants expressing thyroid stimulating hormone receptor (hTSHR) or thyroid stimulating hormone receptor-extracellular domain (hTSHR-ECD) and transformed plants. In addition, the present invention provides a method for preparing hTSHR or hTSHR-ECD from the transformed plants. As the present invention utilizes eucaryote-expression systems, the final products, hTHSR or hTHSR-ECD, are very likely to be secondarily modified and the convenient cultivation of transformed plants will allow for the mass production of human autoantigen, hTHSR or hTHSR-ECD.

1. A method for preparing transformed plants expressing thyroid stimulating hormone receptor (hTSHR) or thyroid stimulating hormone receptor-extracellular domain (hTSHR-ECD), which comprises the steps of:
(a) transforming plant cells with the following polynucleotide sequences: (i) a polynucleotide sequence encoding hTSHR or hTSHR-ECD; (ii) a promoter that functions in plant cells to cause the production of an RNA molecule operably linked to the polynucleotide sequence of (i); and (iii) a 3′-non-translated region that functions in plant cells to cause the polyadenylation of the 3′-end of said RNA molecule;
(b) selecting transformed plant cells; and
(c) obtaining transformed plant by regenerating said transformed plant cells.

2. The method according to claim 1, wherein said plant is Nicotiana tabacum, Cucumis melo, Curcumis sativa, Citrullus vulgaris or Brassica campestris.

3. The method according to claim 1, wherein said transformation is performed with an Agrobacterium transformation system.

4. The method according to claim 3, wherein the Agrobacterim transformation system is an Agrobacterium tumefaciens-binary vector system.

5. The method according to claim 1 further comprising recovering hTSHR or hTHSR-ECD from the regenerated transformed plant.

6. A transformed plant prepared by the method of claim 1 which expresses hTSHR or hTHSR-ECD.

7. A method for preparing thyroid stimulating hormone receptor (hTSHR) or thyroid stimulating hormone receptor-extracellular domain (hTSHR-ECD), which comprises the steps of:
(a) transforming plant cells with the following polynucleotide sequences: (i) a polynucleotide sequence encoding hTSHR or hTSHR-ECD; (ii) a promoter that functions in plant cells to cause the production of an RNA molecule operably linked to the polynucleotide sequence of (i); and (iii) a 3′-non-translated region that functions in plant cells to cause the polyadenylation of the 3′-end of said RNA molecule;
(b) selecting transformed plant cells;
(c) obtaining transformed plant by regenerating said transformed plant cells; and
(d) recovering hTSHR or hTHSR-ECD from said transformed plant.

8. The method according to claim 7, wherein said plant is Nicotiana tabacum, Cucumis melo, Curcumis sativa, Citrullus vulgaris or Brassica campestris.

9. The method according to claim 7, wherein the transformation is performed with an Agrobacterium transformation system.

10. The method according to claim 9, wherein said Agrobacterim transformation system is an Agrobacterium tumefaciens-binary vector system.

11. A transformed plant prepared by the method of claim 2 which expresses hTSHR or hTHSR-ECD.

12. A transformed plant prepared by the method of claim 3 which expresses hTSHR or hTHSR-ECD.

13. A transformed plant prepared by the method of claim 4 which expresses hTSHR or hTHSR-ECD.

14. A transformed plant prepared by the method of claim 5 which expresses hTSHR or hTHSR-ECD.
